(19) United States
(12) Patent Application Publication
(10) Pub. No.: US 2023/0320586 A1
(43) Pub. Date: Oct. 12, 2023

(54) POLICE ASSISTANCE DEVICE AND METHODS OF USE

(71) Applicant: Corey Joseph Brewer, Washington, GA (US)

(72) Inventor: Corey Joseph Brewer, Washington, GA (US)

(21) Appl. No.: 18/299,515

(22) Filed: Apr. 12, 2023

Related U.S. Application Data
(63) Continuation of application No. 17/569,499, filed on Jan. 5, 2022, now abandoned.
(60) Provisional application No. 63/133,931, filed on Jan. 5, 2021.

(51) Int. Cl. A61B 3/113; A61B 5/00; G06V 40/20; G10L 15/22; G06V 40/18; H04N 23/50

(52) U.S. Cl. CPC A61B 3/113; A61B 5/4863; G06V 40/20; G10L 15/22; G06V 40/18; H04N 23/50; G06V 2201/05

(57) ABSTRACT

A device may provide a police assistance device to a person. The police assistance device includes hardware for interacting with a person independent of a police officer such as a processor, memory, a display, a camera, a microphone, and a speaker. The devices and methods may connect the suicide attempter with a chosen contact or a mental health provider. The devices and methods may help with traffic stops. The devices and methods may help with weapon detection.

### CROSS REFERENCE TO RELATED APPLICATION

This application is a continuation of and claims benefit of U.S. non-provisional patent application Ser. No. 17/569,499 filed on Jan. 5, 2022, which claims the benefit of U.S. provisional patent application Ser. No. 63/133,931 filed on Jan. 5, 2021.

### INCORPORATION BY REFERENCE

The entire contents of U.S. non-provisional patent application Ser. No. 17/569,499 filed on Jan. 5, 2022 and U.S. provisional patent application Ser. No. 63/133,931 including the respective specification, claims and drawings are incorporated herein in their entirety.

### TECHNICAL FIELD

The present invention relates to Digital Evidence Collection, Video Recording, Audio Recording, Digital Display, Visual and Audio Instruction Guide.

### BACKGROUND

Police officers deal with many situations on a day-to-day basis. Commonly, officers are involved with testing drivers for driving under the influence (“DUI”) or driving while intoxicated (“DWI”) in what is known as a sobriety test. There are three common field sobriety tests: The Horizontal Gaze Nystagmus (“HGN”) Test, “The Walk and Turn” Test, and “The Standing One Leg” Test. The officer conducts a test, and then issues, if appropriate, a citation and perhaps a summons to a court date. While in court, the officer will be called upon to provide details of the testing, which will be heavily scrutinized by a defense attorney and a judge.

Another situation the police officers deal with his being called to the scene of a suicide attempter. For the purpose of this specification, “suicide attempter” will be the term used for someone contemplating suicide but that is still alive. “Death by suicide” is the term that will be used for a victim that has died. Death by suicide is a global mental health pandemic and claims far too many lives each year.

### SUMMARY

The police assistance device is a standalone operating device that can be equipped, among other things, with a digital display screen, speakers, a camera, a portable power supply with an operating system onboard the device, which runs the device and the preloaded program, which consists of digital displays and audio instructions for the Horizontal Gaze Nystagmus (HGN) test found in a Standardized Field Sobriety Test. The device can provide visual and audio instructions on how to perform the “WALK and TURN” and “The ONE LEG STAND” sobriety tests while recording the events and storing them for evidentiary purposes. The device can be a closed unit encased in a durable weather resistant casing, equipped with a handle for easy carrying. The unit can be stabilized by a device base such as tripod system that extends from the bottom of the casing and retracts into the bottom when use is over, which makes for easy set up and clean up anywhere in the field. The device can be utilized in an array of situations including different geological areas and in all weather conditions. The device can be used as a Mobile Field Sobriety Testing unit which can be deployed by law enforcement, Military, TSA, DOT, FRA, or any operator wherever they are in the field. The device utilizes the onboard display to display instructions, from worded instructions to visual/video instructions. The speaker system can audibly give directions in conjunction with what is displayed on the screen. The camera system will record the individual's response to the instructions given and their interaction with the device and store the audio and video recording, to a storage device within the operating system. The instructions provided by this device can be field sobriety instructions, standard traffic stop instructions and suicide prevention aids which will also be available through this device helping to bridge the barrier between law enforcement and the public as well as elevate any language barriers or personal disabilities, such as deafness or blindness that members in the field may encounter. Methods are described that can follow these instructions. Additional methods for weapons detection and deterrents are described.

Additional features of the devices and methods will be described and will form the subject matter of the claims appended hereto if recited or added through amendment. In this respect, before explaining at least one embodiment of the device, it is to be understood that the devices are not limited in its application to the details of construction or to the arrangements of the components set forth in the following description or illustrated in the drawings.

### BRIEF DESCRIPTION OF THE DRAWINGS

The accompanying drawings, which are incorporated in and constitute a part of this specification, illustrate examples, and together with the description, serve to explain the principles. The drawings are not to scale unless otherwise indicated. For the purposes of these drawings, directions are indicated from a front of device orientation. For example, a “left” speaker from the front side is still a “left” speaker looking at the device from the rear.

FIG. 1 is a front upper perspective view of a first example embodiment of the device with the tripod retracted.

FIG. 2 is a front view of the same embodiment of the device with the tripod extended.

FIG. 3 is a rear view from the upper left perspective of the same embodiment with the tripod retracted.

FIG. 4 is a bottom view of the same embodiment depicting the retractable tripod slot and retracted tripod therein.

FIG. 5 is a right-side view of the same embodiment showing a speaker and data ports.

FIG. 6 is a left side view of the same embodiment showing another speaker.

FIG. 7 is a rear view of the same embodiment.

FIGS. 8A-8C depicts an example square indicator/stimulus on the device's display at different positions during an example embodiment of a Horizontal Gaze Nystagmus test being conducted by the device.

FIG. 9 depicts an example embodiment of a person's video chat with a third party on the display.

FIGS. 10A-10B depicts an example prompts on the display used to ascertain the language of operation. FIG. 10C is an example of the device getting consent from a person.

FIGS. 11A-11C are prompts of an example embodiment of instructions or prompts related to a weapon detection by the device.

FIG. 12 is a flow chart of an example method of conducting an HGN field sobriety test on a person.

FIG. 13 is a flow chart of an example method of determining and using a preferred language for a field sobriety test.

FIG. 14 is a flow chart of an example method of detecting and responding to a weapon.

FIG. 15 is a flow chart of an example method of data gathering by the device.

FIG. 16 is a system schematic view of the electronic components and related devices.

### DESCRIPTION OF THE INVENTION

Documentary evidence of sobriety testing is critical. The current system relies on officer inputted notes that are likely not contemporaneous with the testing. Distractions are common in the nature of police work, such as getting called to handle something else. Mistakes in testing or in recording evidence made during the testing can be fatal to the case of catching someone that is truly intoxicated above a legal limit, or vice versa, can wrongly implicate an innocent driver. An object of the devices and methods herein is to reduce mistakes made by officers during testing for DUI or DWI. A further object of the devices and methods herein is to capture better evidence during the testing that can be proven in court later. Body cam footage is not accurate for the HGN test.

Another object of the devices and methods herein is to increase officer safety by removing officers from potentially volatile and/or dangerous situations. Another object of the devices and methods herein is to reduce the number of suicides by removing people with weapons at the scene of a de-escalation attempt. Another object is to reduce the potential influence of the motions by an outsider on the suicide attempter. Another object is to increase communication ability with test subjects were suicide attempters that speak different languages from those of the test administrators. Another object of the devices and methods herein is to record interactions between officers and suicide attempters and test subjects so that way the officer can focus on what is happening in the present, and not as much focus is needed on recording the evidence that was created in the past. Another object of the devices and methods herein is to detect the presence of a weapon and/or escalation of a weapon use. Another object of the devices and methods herein is to be able to de-escalate dangerous situations with non-lethal alternatives when a weapon is detected through the use of on device methods, such as a strobe light 205 or non-lethal dosage in a tranquilizer dart. For less serious situations, an emission of Cannabidiol (CBD) aroma, a release of nitrous oxide (aka laughing gas) or other calming vapors, essential oils or pleasant scents could help to diffuse the immediate tension of a crisis situation. Another object is the ability to involve 3rd parties in de-escalation attempts, e.g., a family member of a suicide attempter, that may be able to build a better rapport with the suicide attempter or the test subject. A really simple solution is to offer to play a movie or other television or music to buy time until the person calms down (e.g., drug withdrawal) or offer a cigarette from an optional storage compartment with a dispenser and lighter on the device. Another object of the devices the methods herein is to use artificial intelligence to detect when conditions are present that may cause false positives and test results. Another object of the devices and methods herein is to use artificial intelligence to pretty analyze the results and make recommendations to an officer and/or a judge.

Described herein is a police assistance device (referred throughout this specification as “the device.” The same or similar embodiments of the device can operate as both a mobile field sobriety testing and evidence gathering device, a suicide attempter response device, and other common situations such as a traffic stop that occur frequently for a police officer. Also described herein are methods of using the device in these different situations.

Because the device has multiple uses, the following terms will be interchangeable throughout the specification.

The device will provide detailed instructions via visual and audio display, to individuals interacting with the device. The device will be stationed in front of the individual and turned on, activated or self-starting. The device will instruct the individual to carry out a series of tasks in a sequence. The device will provide instructions to the individual through a display 20 and audio recording (or real time text reading) through speakers 70, 80. While the individual performs the tasks, the device will capture an audio and video recording of the performance. The device will store the recordings and allow removable memory devices to capture and store the audio and video recordings of the performance which can be stored as physical evidence.

The device streamlines digital forensic evidence in relation to visual and audible instruction guided interactions between civilians and Law Enforcement. The device will accurately document and store evidence related to tasks performed by individuals instructed by the device. The storable evidence will streamline cases, avoid officer safety hazards, and better equip Law Enforcement personnel to accurately detect impaired individuals. The device's ability to record audibly and visually will prevent inaccurate documentation of facts and process the information received in a better evidentiary manner.

The “Police Officer” providing the device and setting up the test will be interchangeable with “Test Administrator.” The test administrator is the person who is responsible for deploying the test. The test administrator could be an officer, military personnel, a teacher, or any other person or presence. The test administrator does not have to be or remain on site. Police officer can also be understood to be other government officials, mental health professionals, or others who may be able to benefit from usage of the test described herein.

The “Person” that would normally be engaging with a police officer and receiving the test or other prompts and interactions with the device is also “Test Subject” or a “Suicide Attempter.” While it should be appreciated that these individual groups can be similar or mutually exclusive, the device can interact with persons so the described interactions in the specification should be understand as being able to be performed on any of these individuals.

An object of the device is to provide visual and audio instructions that are interactive while creating documentary evidence (audio and visual) and proof of instruction being given. Presently there are no devices utilizing the three elements of visually displayed instructions paired with audio instructions, while being audibly and visually recorded.

An object of the device is providing a complete unit utilizing visual and audio instructions in sequence, while utilizing video and audio recording devices to capture digital evidence of the instruction being provided by the device. Digital display and audio will be utilized by the device to provide instructions to the persons interacting with the device. The device will capture a detailed description of the person receiving the instruction via visual and audible recordings.

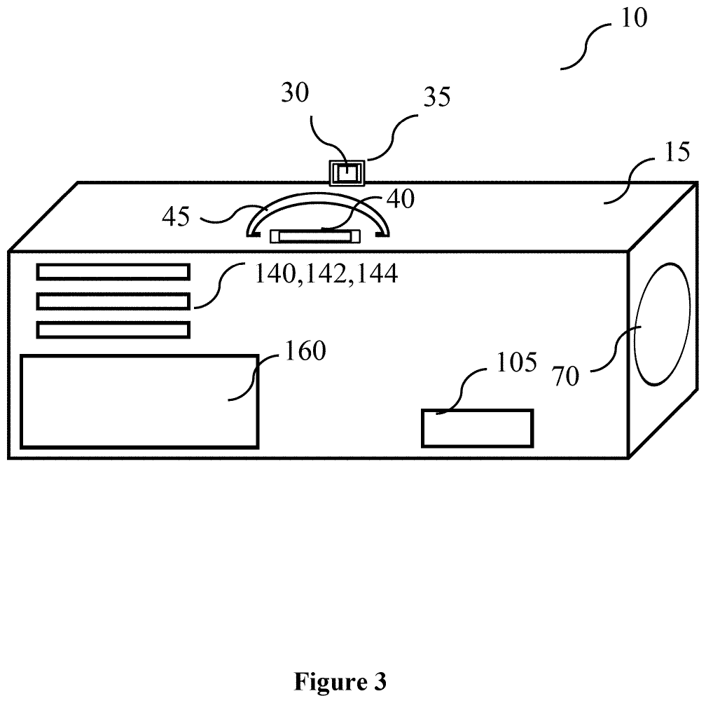

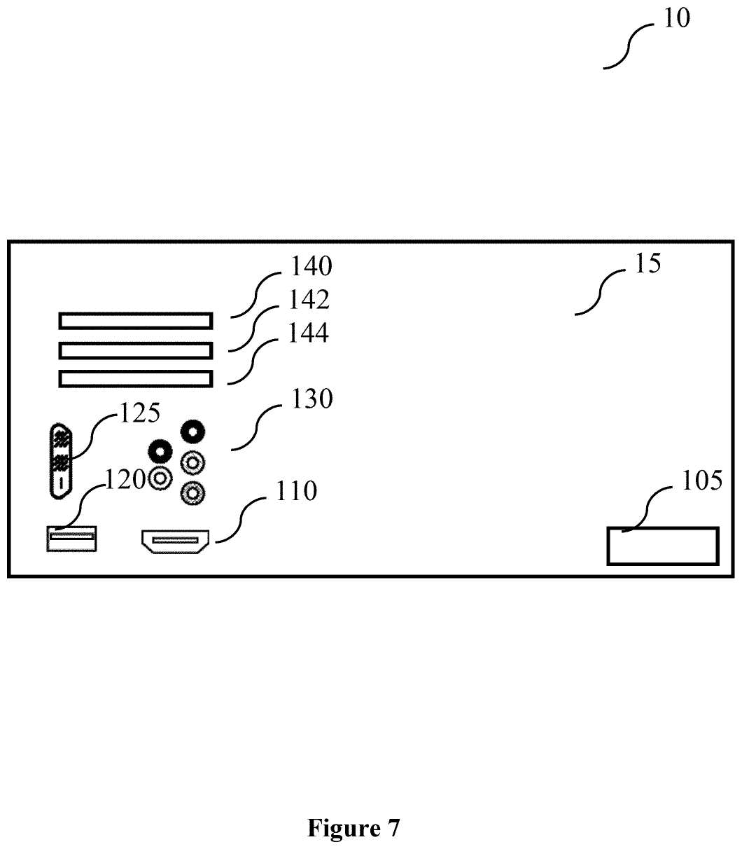

FIGS. 3 and 7 depict the rear of the device. The device can be outfitted with a variety of wired or removable media connections to other devices. The device can have any number of ports for outputting data (USB types A-C, SD Card, Micro SD cards, RJ-45 ethernet), image/video (S-Video, VGA, HDMI, DVI), audio (telephone jack, 3.5 mm audio jack) and other typical ports. As shown in the figures for the first example embodiment of the device, these connections can be in a connections area 160 or located at various locations on the device. Some potential connections that are shown include USB (Speaker Side) 85, SD Card Slot 90, Charging Port 105, HDMI 110, USB (Back) 120, DVI Port 125, 2-Channel Audio and Component Video 130. Processor 310 may be disposed of in communication with one or more input/output (I/O) devices via an I/O interface (not shown). I/O interface may employ communication protocols/methods such as, without limitation, audio, analog, digital, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, etc. A person of skill in the art would recognize that many combinations of connections can address the needs of the people needing to use the device.

The rear of the device also shows ventilation slots 140, 142, 144 that are used to dissipate heat generated within the device.

The device has an outer case 15 that is robust enough for daily use and vehicular transport. Metal or hard plastic work for this purpose. Any other material that can provide this robustness is suitable.

It is possible to use waterproof components such as waterproof speakers, waterproof cameras like the Olympus® Tough® camera, and outdoor displays designed to be exposed to the elements. More rugged components create a device that can take more abuse. It is also can be insulated and adapted to different climates.

The outer case 15 can have a sealing system (not shown) to waterproof all potential water ingress opportunities in the outer case and its connections with its components for any components that are not themselves waterproof. The outer case may include clear coverings for the display or camera so that less expensive, non-waterproof components can be used and lower the overall device cost. A very simple sealing system could be a cover or a poncho that is only put on when the device is exposed to the elements and cannot be stowed away in a protected area.

The device optionally has at least one handle 45 for carrying the device. It is shown on top of the device but can be elsewhere on the device provided it doesn't block the camera or the display from the test subject or otherwise interfere with the device's functionality.

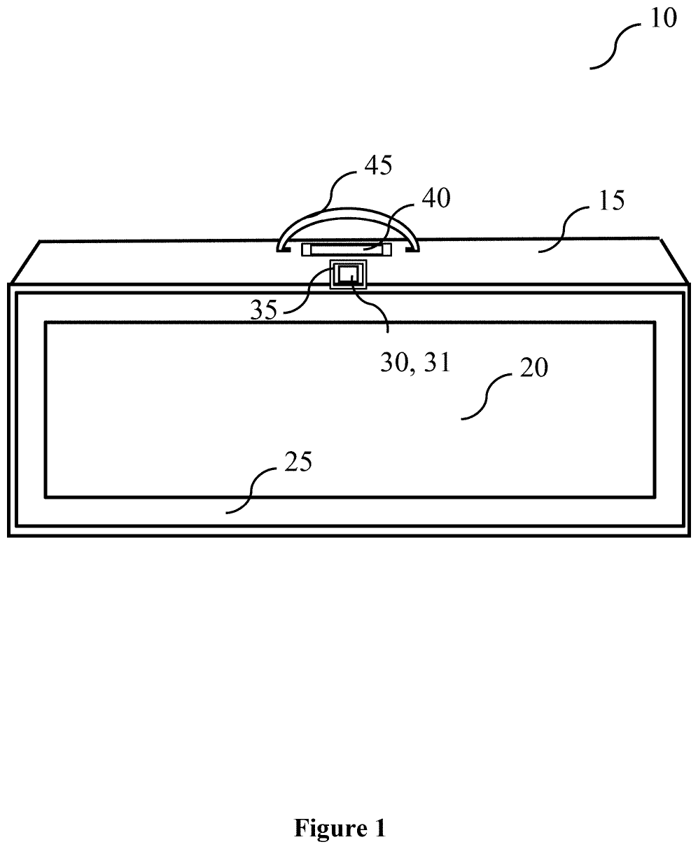

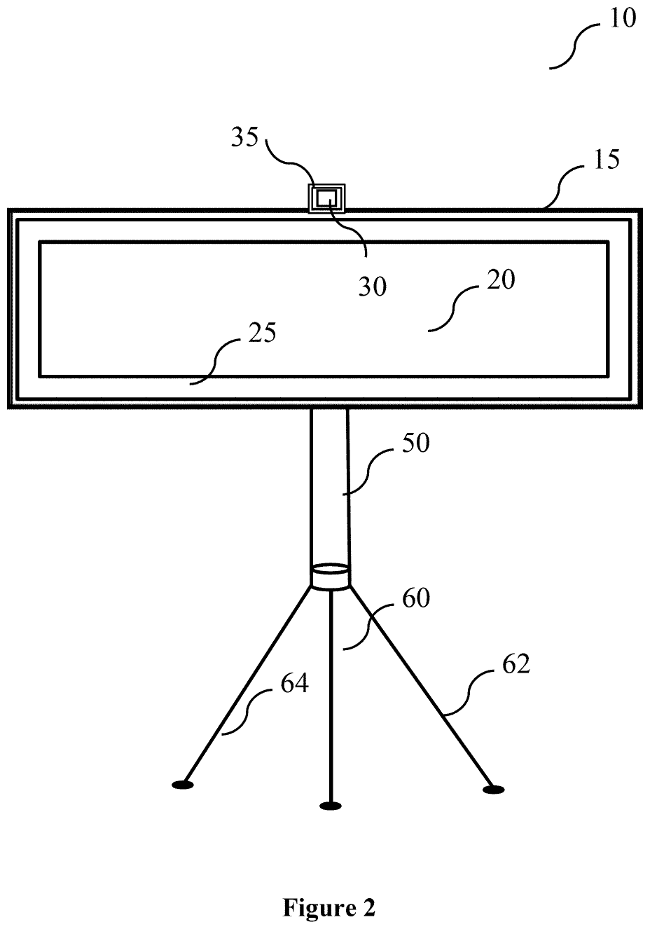

It is convenient to hide a release leaver 40 underneath the handle 45. The release lever 40 or a similar mechanism is used to move the device base from a collapsed state into an expanded state. In the embodiment of FIGS. 1-6, the collapsed state is a fully retracted tripod 50 with legs 60, 62, 64 condensed in a base receiving area 400. The release lever 40 could be a button actuator, a hydraulic/pneumatic system, a pressure release system, or any other technology that accomplishes the same purpose. Additionally or alternatively, the device base could have enough resistance that force alone is enough to move the device base without a release being needed at all.

The device can be operated by a battery (not shown) or through a power/charging cable connected to charging port 115. For the battery embodiments, the device can have a charging port and permanent battery(ies) and/or a swappable battery(ies) (not shown). The power cable connected to charging port 115 at its other end can connect with a wall outlet, a vehicle cigarette lighter or other port with a power. The device can be recharged using on an on-device or off-device solar cell (not shown).

The display 20 can take up the entire front of the device or it can be a smaller display with monitor casing around it as depicted in the figures. It is held in and protected on its edges by monitoring casing 25 but could be held in or protected by other means.

An optional rear or side display (not shown) outputting the camera footage and/or the testing screen can facilitate a test administrator's confirmation that the testing is configured correctly and will yield an accurate result.

The device can have wireless communication capabilities such as Bluetooth, WiFi (IEEE 802.n/b/g/n/x), cellular service (e.g., 3G, 4G, 5G, GSM, LTE, WiMax, CDMA, HSPA+ or the like), radio, satellite, and the like.

The device can be designed to operate well in remote environments with minimal connectivity which could be needed in very rural areas. On the alternative flip side, the system can be designed with the latest functionality for communicating with the cloud and databases to provide real time artificial intelligence analysis conducted on device or remotely and communicating test results in real time and communicating with the officer in real time and recording devices on archival systems in real time.

As the stimulus in the HGN sobriety test should be approximately at the test subject's eyebrow height, the camera 30 and light 35 should sit in proximity to the display, so they are not too far or at too large of an angle to be able to capture accurately the test results of the subject's eyes. The camera 30 can be mounted on the outer case 15, in part of the monitor casing 25, integrated visibly or invisibly with the display 20, or attached to the device in some other way. It is possible for the device to be wired or wirelessly connected to an external camera and lighting source and capture data therefrom.

The device has at least one camera 20 capable of capturing the footage. The camera has a sensor (e.g., CMOS) that allows it to capture visible light. Additionally or alternatively, the camera can be any eye position detection device even if it does not rely on pixels. The footage can be captured as still images or video images or positions.

The device can use a light 35 such as a ring light, light array setup, diffuser, or any other lighting tool to get the quality of light to get a clear test result. A smaller amount of more efficient lighting would help minimize battery power loss, when applicable. The display itself can provide the light also, such as around the outer edge. A microphone 31 can be part of the camera 30 assembly or separately found on the device to record audio of the subject's response to prompts, the testing, and the test results.

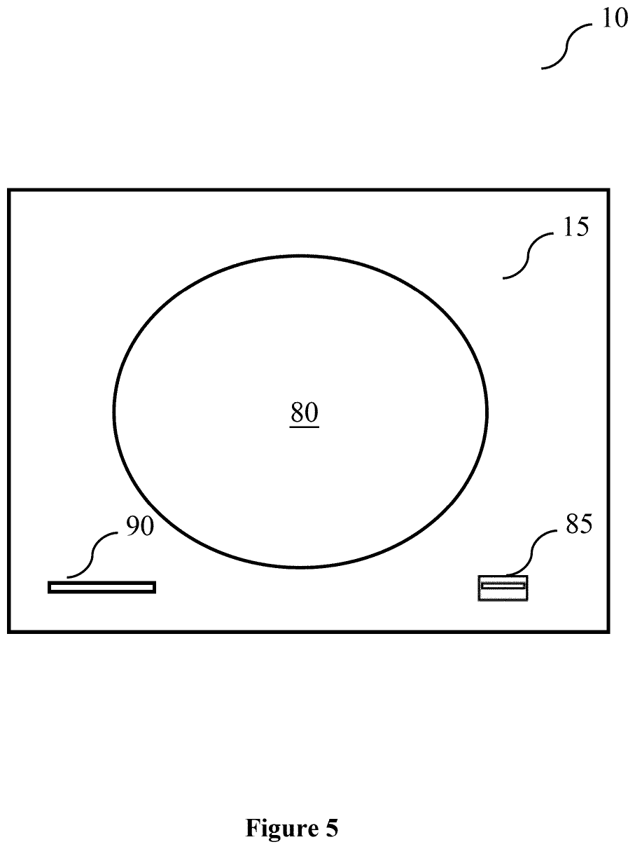

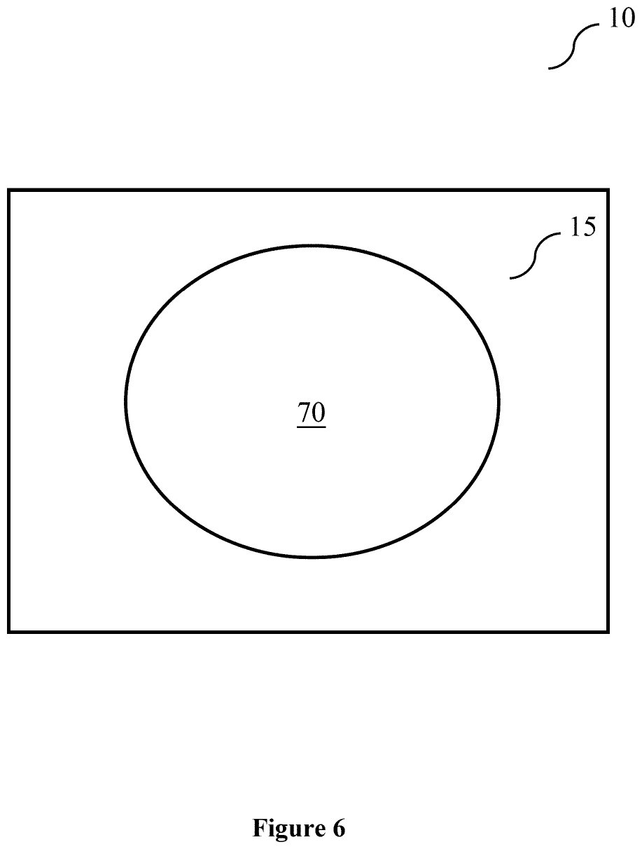

It is possible to operate a testing device through visual prompts only whether using a touchscreen or another user interface for entering information. However, it is appreciated that audio prompts and instructions facilitate the testing. Off-the-shelf speakers 70, 80 depicted in FIGS. 5 and 6 capable of transmitting audible instructions to the test subject will work. Different volume levels can be helpful to accommodate different hearing abilities. Speakers 70, 80 that are water resistant or waterproof offer additional functionality in adverse weather conditions. The depicted embodiments have two speakers in a boombox-type setup, but a person of skill in the art would realize that many other combinations and variations would achieve a similar goal. Additionally, a loudspeaker (not shown) could be added to the device to provide other functionality such as remote crowd control, warning messages, playing music or other instances when having loud messaging is appropriate. The speakers 70, 80 could be on the device or could operate remotely through a headset or a sound bar type of setup.

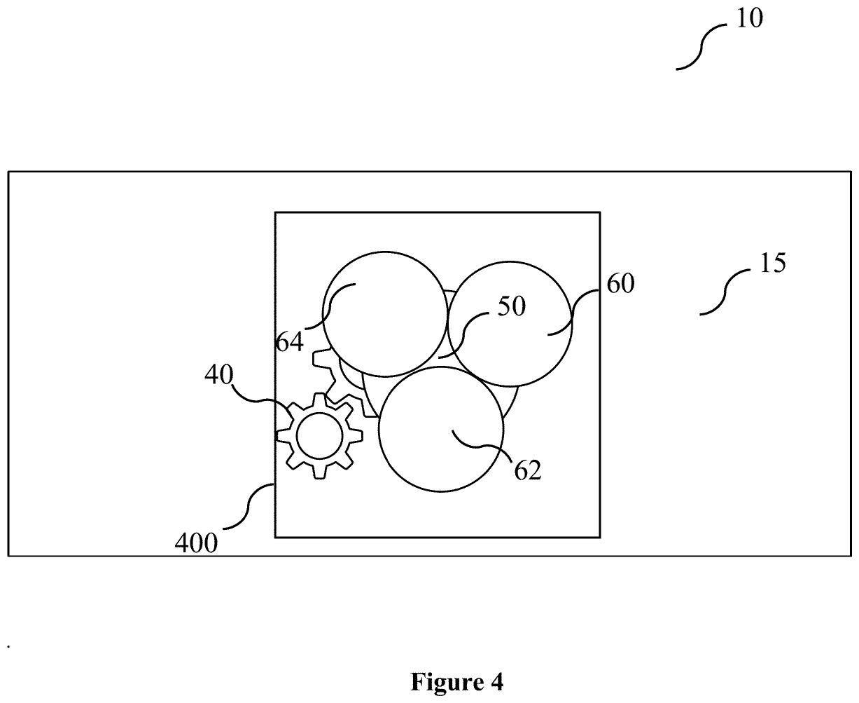

The device has a device base intended to support the device and a height adjustment system. The depicted embodiment of the device has a retractable tripod 50 that stows away in a device base receiving area 600 in the shape of the device base on the bottom of the device 10 to make the device portable. The tripod legs 60, 62, 64 are adjustable and can be used to adjust the height of the device 10. Here, the tripod 50 both supports the device as its device base and has a height adjustment system using the extendible tripod legs 60, 62, 64. FIG. 4 depicts the tripod fully retracted inside the device base receiving area 400. A gear opposite and activated by release lever 40 can release the tripod from a fixed position inside the device. Other embodiments of a device base are possible that may or may not have the height included such as wheels, legs, tracks, rollers, or other terrain movement devices. For example, the device can be a robot with a track mobility and height adjustment system such as Johnny 5 from the Short Circuit movie franchise. Johnny 5 is incorporated by reference herein. The mobility system lets the device move along the ground, up stairs, or otherwise to get to the location the device needs to be. The mobility system can be controlled by a control system on the device or remotely through a joystick or an app. In some embodiments, like the tripod, the device base can still inside of the device and be retracted when needed. In other embodiments, the device base is removable and re-attachable to the device so that different device bases can be used for different needs. In still other embodiments, the device base is not intended to be removed or stowed away.

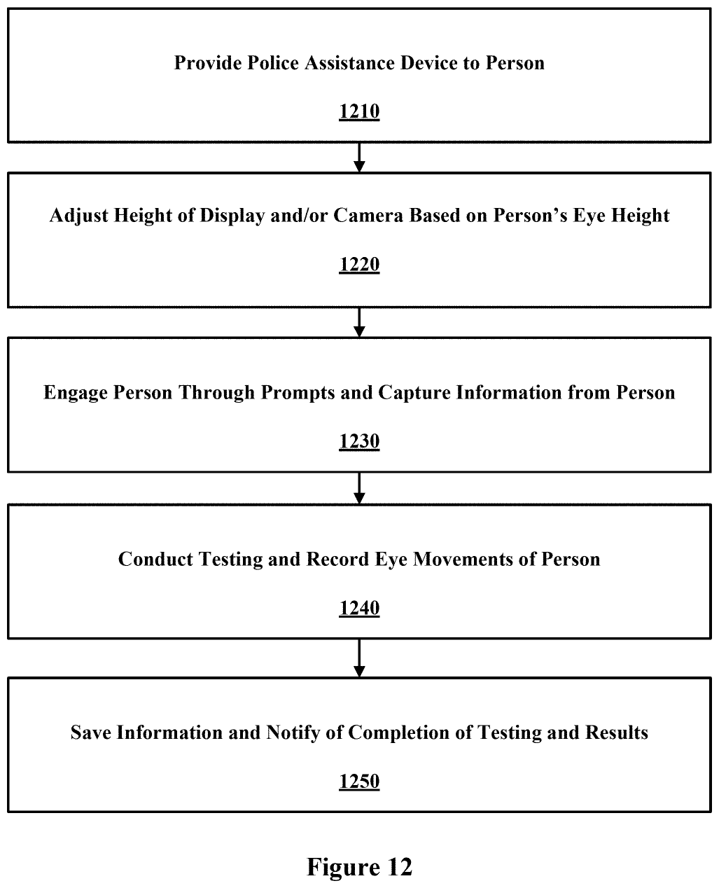

FIG. 12 is a flow chart of an example method of conducting an HGN field sobriety test. First, the police officer or test administrator provides the police assistance device to the test subject in step 1210. Step 1220 involves adjusting the height of the display and the camera to be near the person's eyes. Step 1230 involves engaging the person through prompts, preferably in their language, and capturing information from that person using the camera, the microphone and or touchscreen. Then the HGN test is performed in step 1240, recording the eye movements of the person as further discussed below. And then in step 1250, the information is saved for evidence for a court proceeding.

The device does not need to have optional machine-driven mechanical movements, as shown in the first example embodiment. The test administrator can use a tripod base 50 and legs 60, 62, 64 to adjust the system upward or downward to the desired height that is optimized for testing the user with the HGN field sobriety test, or merely for interacting with the suicide attempter. The device with the tripod can be static with tripod legs or it could be on wheels to be easily moved around. Alternatively, it could have a suction cup or attaching to the hood of a car. Because the hood of a car is not perfectly flat, the height can be adjusted based on the location of the car hood. In the hood embodiment, for example, there may be a little or no device base at all. Additionally or alternatively, there can be an extending neck or other adjustable height mechanism even when attached to the hood to relocate the height to the test subjects eye level more precisely. The system could be a simple ratchet and release system so that way the officer can adjust the height. Or it could be automated with a remote control where other control device or control switch on the device itself where the test administrator could control the height. The height of the device can be tracked as one of the test data Parameters that is recorded.

The height adjustment system is used to adjust at least the display 20 and camera 30 features upwards or downwards. The height adjustment system can be part of the base as previously demonstrated. For example, the display screen 20 and the video recording device 30 can be moved to be closer to the test subject's eyes by extending the tripod legs. The height adjustment mechanism can also be separate from the base. For example, an extendible ratcheting neck can move the display and camera upward, which can be released to lower back the display 20 and camera 30. The speakers 70, 80 and other device components can optionally move up or down with the height adjustment system or can work adequately at their original position. Additionally or alternatively, an automated drive system can control the movement of the height adjustment system. It can be controlled on the device or remotely through a switch or joystick or a smartphone, tablet, laptop app or a web application.

The display 20 can be a monitor, a television, a tablet, a touch screen, a projected image, or other device that can adequately display the messaging, horizontal gaze test, and other images. While energy efficient displays of are the easiest on batteries, any known display technology can be used including LED, OLED, LCD, LCD-LED, quantum dot, e-ink, and cathode-ray tubes. A touch screen display has the advantage that the user or the test administrator can interact with the display using a familiar user interface, and to smart phones and tablets. The display can also be controlled through audio prompts or other buttons on the device or remotely through a connected application. In some embodiments, a very simple display can be used for most functions. In other embodiments, a high-quality display enables more functionality such as video chatting.

The device also has a processor 300, memory 310 and on-device storage 320. The memory 310 stores a plurality of instructions and a processor 300 executes the plurality of instructions. Memory 310 may be a non-volatile memory or a volatile memory. Examples of non-volatile memory may include, but are not limited to flash memory, a Read Only Memory (ROM), a Programmable ROM (PROM), Erasable PROM (EPROM), and Electrically EPROM (EEPROM) memory. Examples of volatile memory may include but are not limited to Dynamic Random-Access Memory (DRAM), and Static Random-Access memory (SRAM). The processor 300 may include at least one data processor for executing program components for executing user- or system-generated requests. The device can also access remote storage on another computing device such as a server, cloud device, workstation, etc.

Processor 300 may include specialized processing units such as integrated system (bus) controllers, memory management control units, floating point units, graphics processing units, digital signal processing units, etc. Non-limiting examples of processor 306 include a microprocessor, such as AMD® ATHLON® microprocessor, DURON® microprocessor OR OPTERON® microprocessor, ARM's application, embedded or secure processors, IBM® POWERPC®, INTEL'S CORE® processor, ITANIUM® processor, XEON® processor, CELERON® processor or other line of processors, etc. Processor 300 may be implemented using mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), etc.

The processor 300 controls the display 20, speakers 70, 80, microphone 31, the display or sounding of the prompts, and in the interaction of the components. Stock prompts can be stored in the device's memory or storage. A universal system bus is used to connect components electronically and communicatively throughout the system. External language prompts can be downloaded as needed, in advance or in real time. It is also possible that the Officer through an app can add custom prompts.

In the embodiment of FIGS. 1-6, Applicant used an ONN® smart tv as the display, a Nexigo® camera, a Powkey® power bank, a Raspberry Pi® single onboard computer, and a Cannon® tripod.

Additionally or alternatively, the device can have a breathalyzer or other chemical sensing abilities to analyze drug. Medical testing often needs to be performed by a valid medical professional, and the device can also schedule appointments with nearby medical professionals so that the test subject can see the medical professional as soon as possible without an escort by the officer, wasting valuable resources. A failure to show up for the test could be penalized to infer the same result is a positive test.

Additionally or alternatively, the device, in addition to the eye movements that are captured during testing, can take at least one snapshot (still image) to verify that the user height matches the system height based on a picture of the users' eyes as taken at the selected height. The device or related systems can have built in eye detection software and adjust the height until the eyes are detected to be approximately level with the system within a safe degree of accuracy to be compliant with the HGN testing standards.

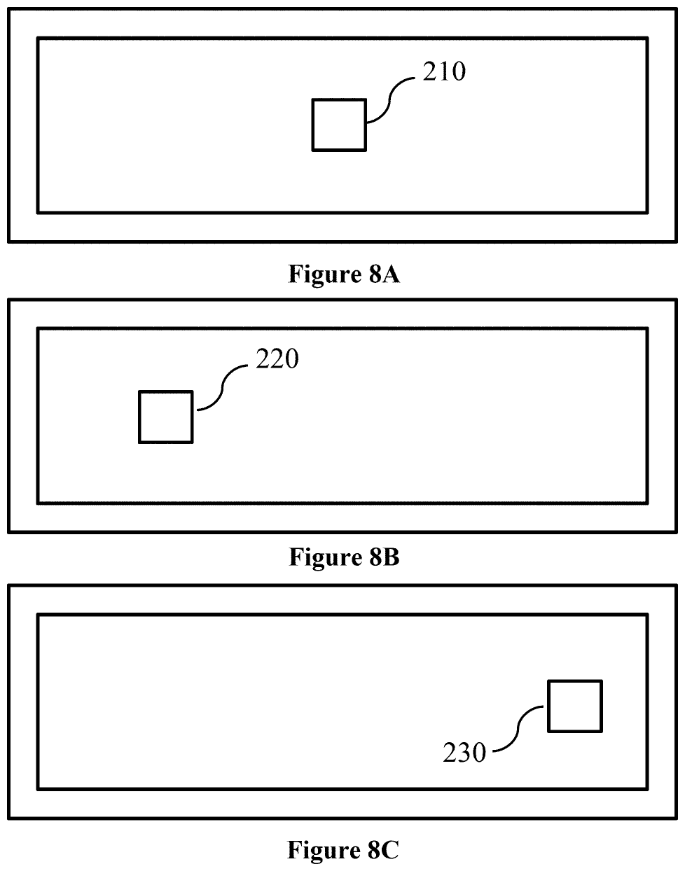

FIGS. 8A-8C shows a moving test indicator in various positions along the display. These items would move in compliance with acceptable HGN sobriety test operating procedures.

Additionally or alternatively, the device can measure boundary allowances. This measurement can be taken from the pupil's travel into the iris of the eye instead of where the pupil should be. When an involuntary jerking happens, the eye will bounce backward from the original path in which it is instructed to move. The iris is the colored portion of the eye, and the pupil is the black center, which could be the point of measurement when in motion any movement not associated with the movement of the moving test indicator will indicate Nystagmus in the eye along with the eye in a stationary position when involuntarily jerking will be indicated by the measured movement.

Additionally or alternatively, the system could also measure distance using a radar sensor or other sensor that's capable of measuring distance. In this way, the device could self-determine that the device is at the appropriate distance to the test subject and can record the distance and the test that it passed the appropriateness distance check before testing began. It can also verify after testing finished that the height and the distance are appropriate.

Additionally or alternatively, if the test subject has corrective lenses, the system can detect that visually, or based on user feedback in response to the systems prompts. The system would record whether or not the user was wearing the glasses for the testing.

Additionally or alternatively, the device can cross check information from a scan of the test subject's drivers license performed by the device using a scanner (not shown). This can be done by the test subject putting their drivers license and vehicle registration in a scanning slot or department or you know against a surface at prior to the testing beginning. Or it can be detected from known databases where the system can cross examine the insurance database to confirm registration in the department of driver services databases for license information.

Additionally or alternatively, the device can capture biometrics for proving the identity of the individual. The device can take the user's fingerprints through a scanner on the device. The fingerprint detection can be run by an officer with rotating fingers under normal standard procedure, or the scanning area where the finger the user places their fingers can be shaped in a finger shape to be able to get accurate sensing or the user can be given instructions on how to use the biometric detection devices correctly and bad attempts prompted for correction. Additionally or alternatively, the system can use retinal scans to positively create an identification match that cannot easily be falsified. This same retinal scan or fingerprints can be confirmed in a court of law. Detected skin color and hair color and other parameters can be objectively tracked and recorded by the device to prove identity as well.

Additionally or alternatively, if the test results indicate appropriateness of a DUI or a DWI, the device can coordinate with judicial scheduling systems to arrange for a court date and deliver/print on the device using optional printer 330 an actual summons to the user of the appropriate time for the user to show up in court and be scheduled in judicial scheduling system or an official warning. With the court scheduling, the user information and the test results can be placed directly into an automatically drafted legal complaint with a verification of the system configuration and all of the test parameters before the test subject was and after the test subject engaged in the test.

Additionally or alternatively, the system can track a variety of other pieces of information that are available that can be measured, inputted, or scanned from nearby systems. For example, information from the police vehicle as to its driving history, the user's speed and directionality from the user's or the officer's GPS, the speed, and the configuration data from speed detection devices like radar guns and other information can all be linked with the device so that way it can track and submit all of this information to the appropriate databases.

Additionally or alternatively, the system can be designed to deescalate the situation with the suicide attempter. First of all, by not having an officer with a gun pulled out or able to be pulled out in response to a suicide attempter's movements, a decrease of the number of fatalities in attempts to deescalate suicide attempters could occur. In addition, by having mechanisms to distract or to diffuse the stressfulness of the situation can help to reduce the number of suicides.

By having an officer be able to place the device in proximity to the suicide attempter and either not enter the scene or to leave the scene to allow the device to prompt and interact with the suicide attempter, the officer safety is enhanced. The device can conduct a preliminary assessment of an individual in crisis. It can have pre-distinguished questions pertaining to individual issues such as mental health crisis, drug and alcohol issues or suicidal crisis. The device can aid in identifying the different crisis areas. The device will get the preliminary information from the individual and ask questions such as last use of alcohol or drugs, thoughts of hurting the individual or others, feelings of anxiousness or depression and ask if the person is seeking assistance with drug and alcohol dependence, depression or homicidal or suicidal thoughts. After the information is gathered the individual can print from the device to contact a mental health specialist for further evaluation via web cam interview or phone-a-friend feature (described below) including communication with the specialist for further instructions and assistance with the particular crisis. The device will not conduct the actual evaluation of the individual rather gather preliminary information to relate (and optionally automatically relate) to the provider and aid in providing a direct access to the professional for further assessment. In fact, if the individual has been missed by the health care system, this preliminary information can help the individual to get back into the system, and the police officer may be able to help the individual receive mental health care.

In addition, the suicide attempter may find the officer at the scene to be comforting, in which case the officer could choose to stay, or the officer could choose to use the device because the officer may add stress to the situation. The device can have prompts that are in different types of voices that can be selected or altered based on the suicide attempter's state. For example if the suicide attempter is not responding well to a voice that is soft and passive, the device can use user prompts that have a much more forced tone that might create a greater response in the suicide attempter and prevent the actions in motion. Alternatively, the system can go from a very direct and forthright voice to a more copacetic voice if the forceful voice doesn't seem to be providing the right kind of response with the user. Additionally, the device can communicate and be controlled by mental health providers own system such as a mobile phone or other login. The mental health provider can not only communicate to the test subject or suicide attempter with their voice or video footage of themselves speaking, they also may be able to control the device to interact with the user in a more friendly way. For example the device can hold out a hand like it's handshaking with the user or put its arm around the user for a sense of comforting. It could create a cup of tea or a cup of coffee using a Keurig like machine on the device. It could offer a cigarette from an on-device storage compartment, a dispenser and a lighter (not shown). It could play sound soothing noises like waves at the ocean or rainfall if the user indicates that such noises might help relax the user. It could play movies, television, or music. It could create an air conditioned or heated environment with optional HVAC features.

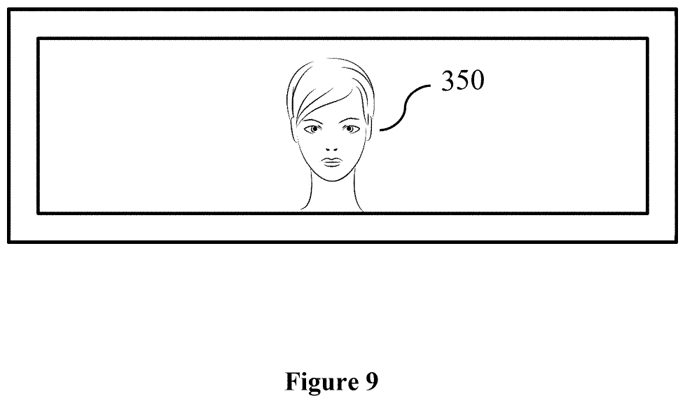

Additionally or alternatively, a phone-a-friend feature can ask the suicide attempter who the attempter wants to speak with and how to contact them. Video or audio connectivity to a mental health care provider or to a family member or other friend of the suicide attempter can help to ground the suicide attempter's emotions in a situation of temporary psychosis, distress, schizophrenia, hysteria or other mental condition and de-emphasize triggers that may have caused a mental health condition to be temporarily present. The device can handle this on its own or with the aid of the test administrator. FIG. 9 shows a video chat occurring on the display with a third party 350. If the user does not have someone to reach out to, the device can default to a mental health care provider hotline. The device may access a variety of modern technologies used for communication such as WhatsApp, Google meet, Facebook chat, Apple FaceTime instant messenger and texting applications, and other later arising communications technologies. The user through the display 20 on the device can have a video chat with this contact 300. The user can also have an audio chat through microphone 31 and speaker 70, 80 on the device. If the user is concerned about what other people may be able to hear who are within earshot of the situation, the device can have a removeable handset on the device through which the user can have a more private conversation with the contact or the mental health care provider. Meanwhile, the system can record the entire conversation and, with appropriate sensors, other physiological symptoms such as heart rate or breathing quickly or other indications of stress or de-stressing, that can be used to help convict or acquit the person.

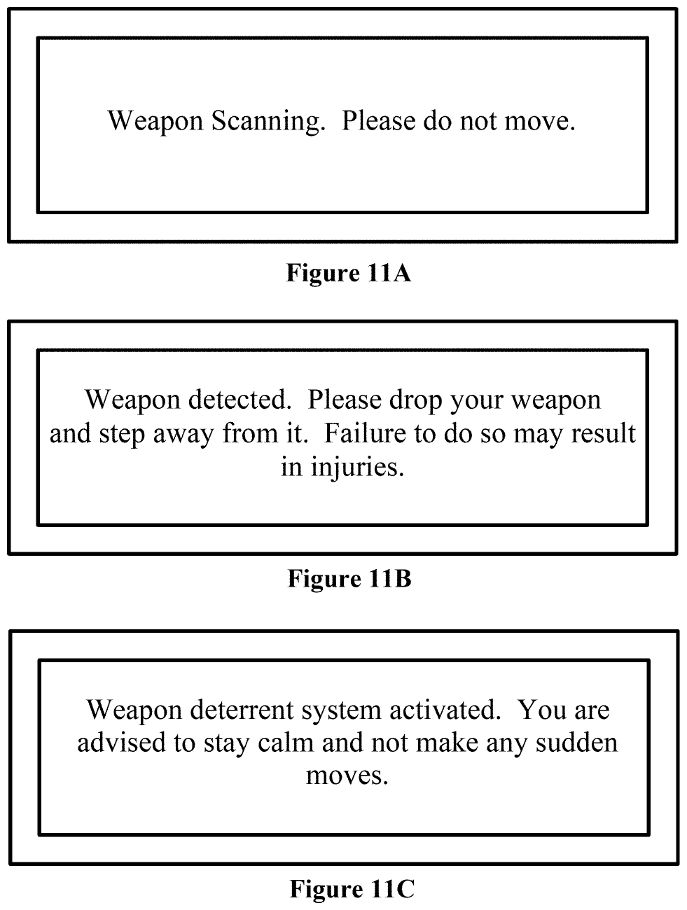

FIGS. 11A through 11C depict some example prompts related to the weapon detection and deterrent system. FIG. 11A is a prompt informing the user a weapon scan is taking place. FIG. 11B is a prompt telling the user that a weapon has been detected and to stand down. FIG. 11C informs the user of an escalation of the weapon deterrent system, which may be perceived together with other actions of the device such as flashing a strobe light.

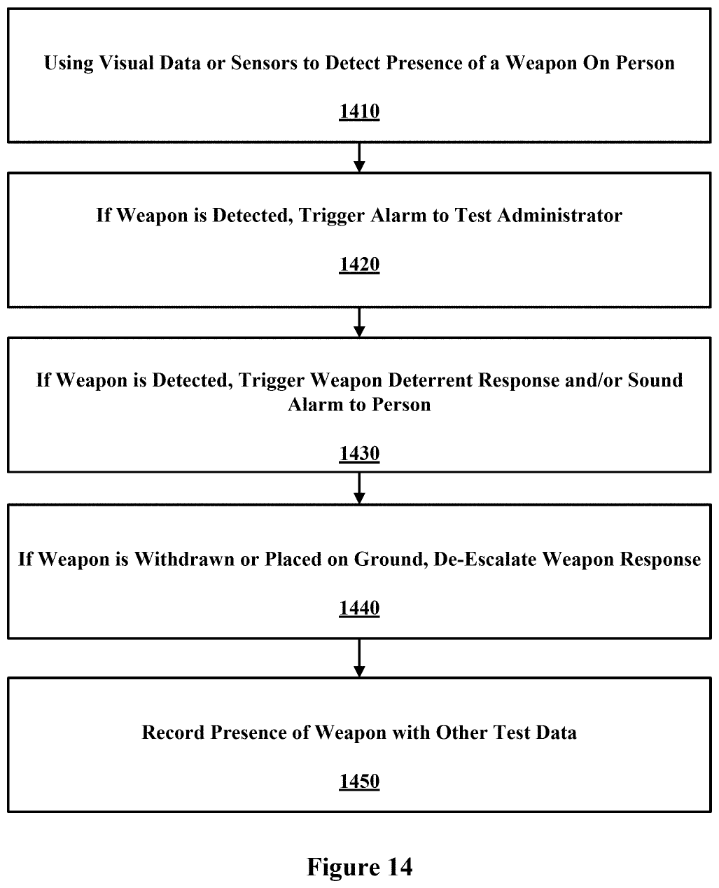

Additionally or alternatively, the device can be outfitted with weapon detection and deterrent systems and used in a way similar to the example method of FIG. 14. Step 1410 uses the sense data including video footage to scan the scene for a weapon. This system can use sensors such as a metal detection device or have real time footage of the scene analyzed to detect whether the suicide attempter has a weapon such as a gun or a knife. Artificial intelligence either on the device or executed remotely through the cloud or a different device on the police patrol car or on a mobile phone can detect the weapon visually. Examples of artificial intelligence gun detection software include zeroeyes, scylla, and evolv. An infrared sensor can assist the visual camera footage to help detect the temperature of the various objects in the scene and correlate with the visual detection to determine something with a different temperature that could indicate a weapon or other object in a user's pocket or in their clothing.

Additionally or alternatively, according to step 1420, if a weapon is detected, the device can trigger a communications response. The officer can be notified privately or through a loudspeaker alarm system. In addition, headquarters or other backup units can be informed that there is a weapon on the scene. The evidence of a weapon being present can also be recorded which could be used in the court case to prove intent with the test being conducted or some other crime that might have been coincident to a DUI or DWI.

Additionally or alternatively, the device can have weapon deterrent features on it. If the device detects a weapon, it can trigger a weapon deterrent response according to step 1430. A strobe light 205 (not shown) can be used to interfere with the person's mental faculties or confuse the person or remind them that this is a police procedure that needs to be taken seriously. If the system detects that the user is using martial arts or aggressive behavior like fighting, this system can respond in the same way or lesser way as it would with a weapon detection with both communication protocols and attempts to halt the aggressive behavior. The deterrent response could be something like a strobe light 205 or a tranquilizer dart, tear gas or a smoke bomb to non-lethally disable the person with the weapon. Any other legal device could be used here as part of a deterrent system.

Additionally or alternatively, to prevent application of heightened stress by the officer in this situation, some of the device's reactions to weapons or other devices could be controlled remotely by individuals not near the scene that may not be influenced as much by the emotional situation at the time.

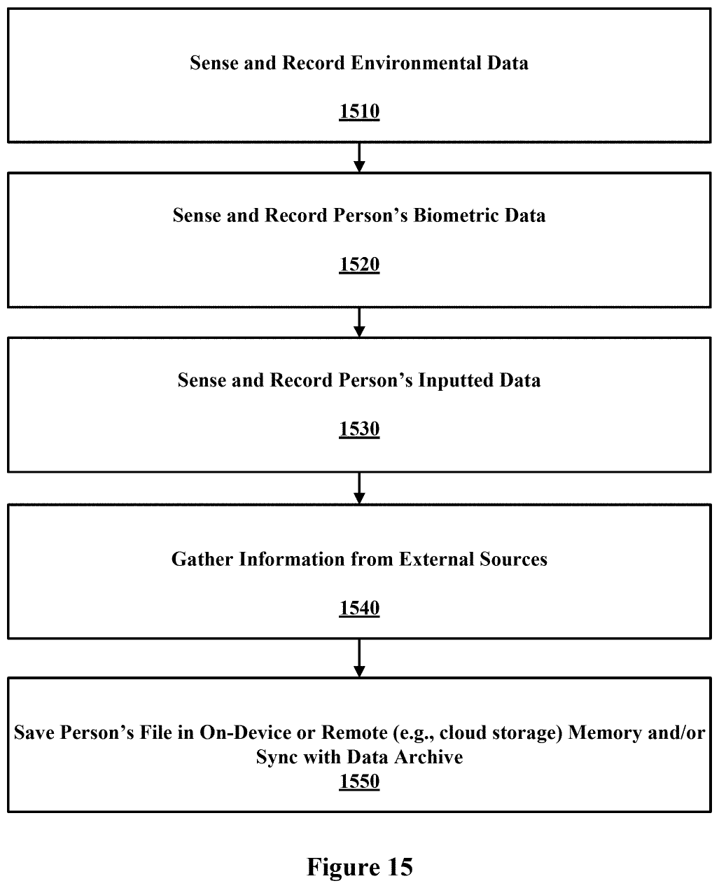

According to step 1440, if the person cooperates and withdraws the weapon or place of the weapon on the ground, the device will de-escalate the weapon response. And in step 1450, the device will record the known data related to the detection of the weapon, combined with other information such as that information gathering in the example method of FIG. 15, and store that information in a file for that person.

If the device is scaring the user and the user has a smartphone, the device can transmit the prompts to the user's mobile phone to do as much as possible in terms of information gathering and recording through the user's handheld device. If HGN sobriety tests or other sobriety tests needs to be administered, that would likely be done on the device itself (although the device could take advantage of the mobile phone's gyroscope and accelerometer during testing or a similarly capable device provided by the officer to be worn or held by the test subject). Through this other connection with the user device, the device should be able to record and capture video and audio of the user through the user's device and control the display of the user device to have a very thorough interaction with the user. The device can also read, capture, or record information about the state of the user's mobile phone, information thereon to prove the user's possession of a phone with certain information on the mobile phone, device identifiers, call logs, and other information, perhaps with the user's consent, which may be used as evidence in appropriate circumstances to corroborate that test results or prove something else.

Additionally or alternatively, the system can continually monitor If the user is hard of hearing, the system can raise its volume or communicate mainly through text on a screen.

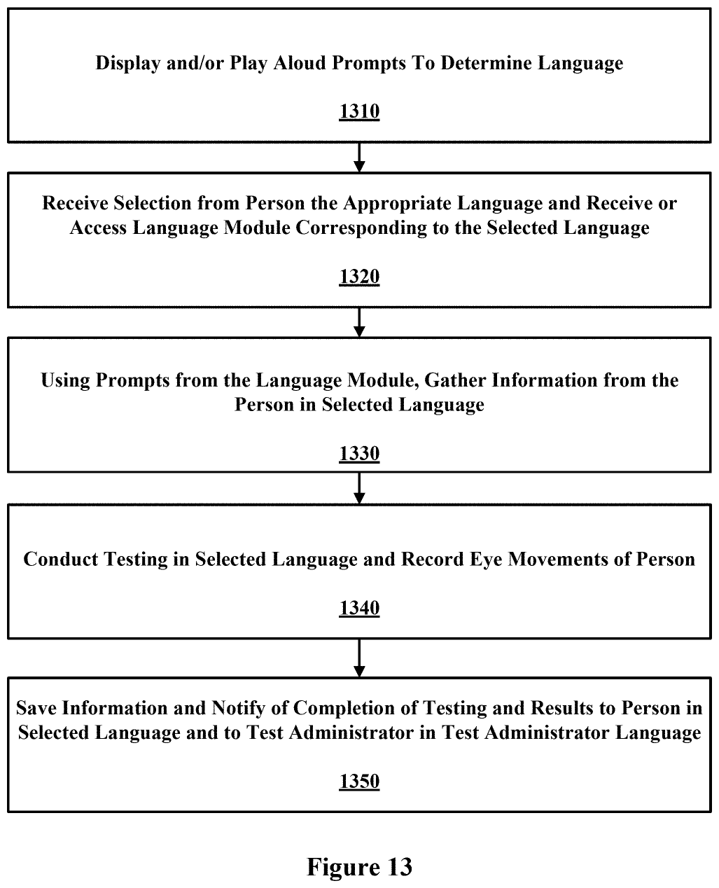

FIG. 13 is an exemplary embodiment of a language input method and/or the execution of software related to language. Unlike an officer that may only have command of a single native language, the device can communicate in multiple languages using pre-recorded prompts, text reading, and real-time translation. When additional languages are needed, the system is capable of a much greater language ability than the police officer. It could have several languages stored directly on the device, or it can access from a remote source such as the cloud language relating to the user. In a hypothetical example, an officer acts as the test administrator and only speaks the languages of English and Spanish. The test subject is a Vietnamese speaker. In this example, due to a language barrier, the test procedures, the test understanding, the result understanding, or some other information may get lost in the communication breakdown. The results may therefore be different if a Vietnamese speaking officer were the person conducting the test of the same Vietnamese speaking test subject. According to step 1310, the device can prompt to the test subject about the test subjects referred language and further adapt the prompts and testing to the selected language in step 1330 and 1340. The appropriate language module is retrieved on the device or from a remote source if such module is not on the device. According to step 1350, the device can speak with the test subject in the test subject's native language and the police officer and the police officer's native language and record the language selection information to generate evidence.

Additionally or alternatively, the system can be configured to gather various pieces of background information such as the time of day and other conditions that and humidity and introduce these into evidentiary safe keeping.

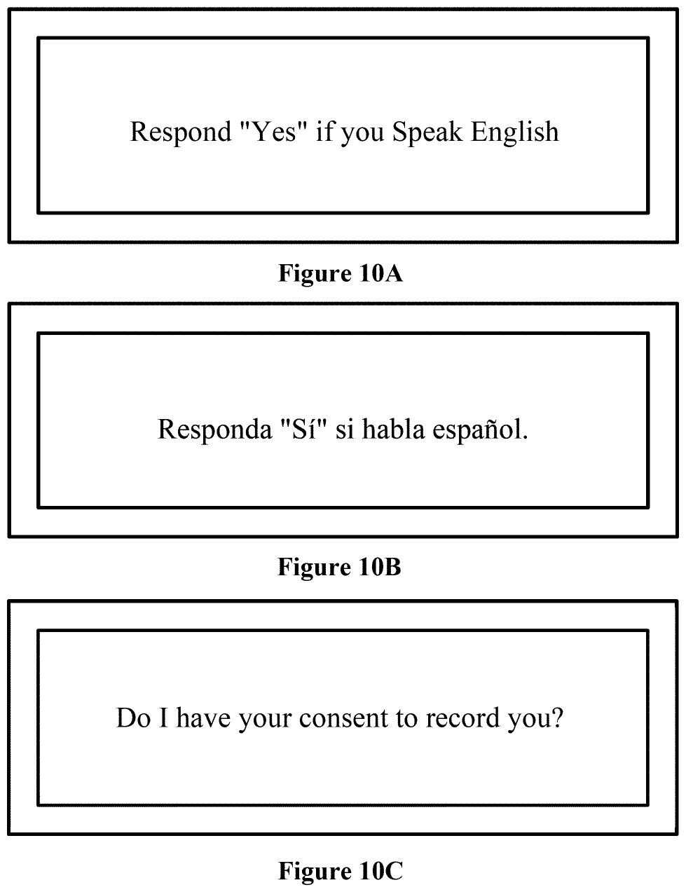

FIGS. 10A and 10B depict prompts on the display asking the user to identify which language that the user prefers to speak. These prompts may also be read allowed. In some jurisdictions, consent may be necessary in order to record the user. FIG. 10C is a prompt request and consents of the user, upon which the device would record the user's answer.

The ability for the device to speak multiple languages with other pleas matter such as routine traffic stops. Basic questions such as asking for license and registration can get complicated when communication or translation issues are present. Also, the fear from the complication and communication or translation can cause individuals and police officers to deviate from their best behavior.

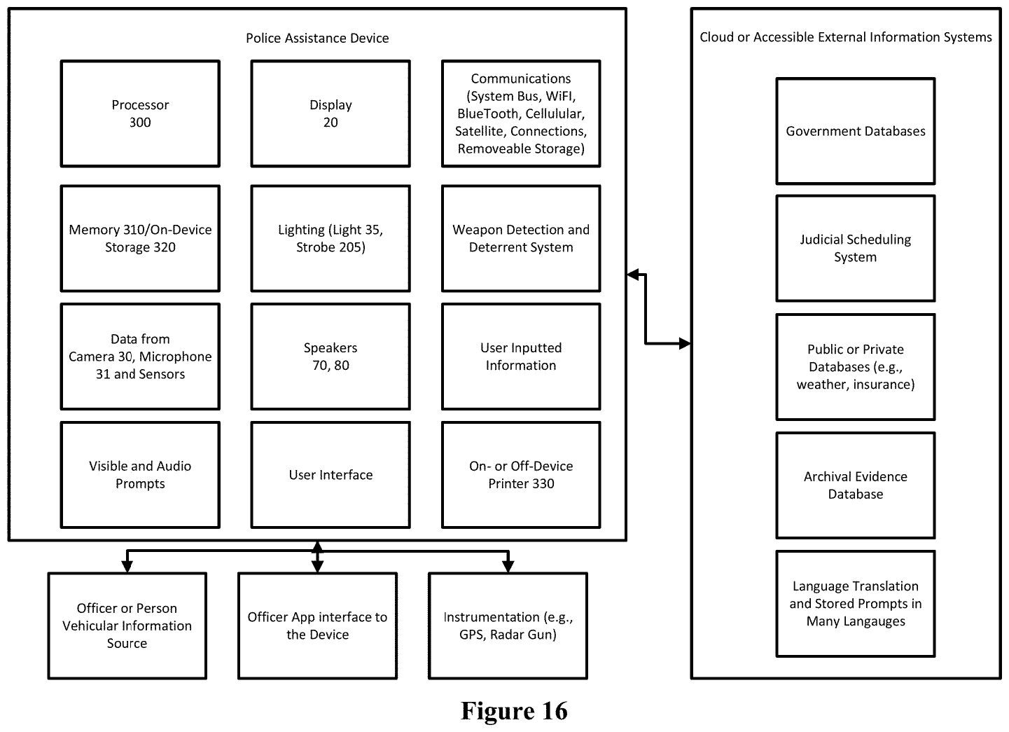

FIG. 15 depicts an example method for gathering data from the area systems and then submitting that data for evidence purposes. Step 1510 involves taking data from the environment such as the temperature and humidity, the date and other information. Step 1520 involves getting information from the test subject which could include identifying information such as skin pigment levels, hair color, retinal scans, fingerprints, facial dimensions, or other information that can be sensed directly from the person. Step 1530 relates to the that are used to get information from the test subject. This information can be entered through the user including by means of nodding yes or no in response to prerecorded prompts, typing on to attach screen, and/or speaking out loud which would be recorded by the microphone. It can also be the individual's driver's license and car registration. Step 1540 includes gathering information from outside sources such as insurance databases, police or FBI records, police scanners, police radar guns, the private-sector information droves like Facebook, or any other available database. The database connections can be set up in advance so that way the saving of the persons information record is contemporaneously done within a data-gathering from the databases. More these databases are shown in FIG. 16. In step 1550, the information from the various sources is gather together and saved in a file connected to the person that can be used as part of the evidence in the person's court proceedings.

FIG. 16 depicts an example embodiment of the hardware architecture and the information flows to and from the place assistance device to and from other systems to be able to gather a complete record for evidence purposes.

There has thus been outlined, rather broadly, some of the features of the device and methods in order that the detailed description thereof may be better understood, and in order that the present contribution to the art may be better appreciated.

Although methods and devices similar to or equivalent to those described herein can be used in the practice or testing of the device and methods, suitable methods and materials are described above. The device and methods may be embodied in other specific forms without departing from the spirit or essential attributes thereof, and it is therefore desired that the present embodiment be considered in all respects as illustrative and not restrictive. Other objects and advantages of the various embodiments of the present invention will become obvious to the reader and it is intended that these objects and advantages are within the scope of the present invention. To the accomplishment of the above and related objects, this invention may be embodied in the form illustrated in the accompanying drawings, attention being called to the fact, however, that the drawings are illustrative only, and that changes may be made in the specific construction illustrated and described within the scope of this application.

It will be apparent to those skilled in the art that various modifications and variation can be made to the disclosed methods, software, hardware, materials, components, and methods herein. Other embodiments will be apparent to those skilled in the art from consideration of the specification and practice of the disclosed inventions. Numerous modifications, changes, variations, substitutions, and equivalents will occur to those skilled in the art without departing from the spirit and scope of claimed invention. It is intended that the specification and examples be considered as exemplary only, with a true scope being indicated by the claims and their equivalents. The benefits, advantages, solutions to problems, and any element(s) that may cause any benefit, advantage, or solution to occur or become more pronounced are not to be construed as a critical, required, or essential features or elements of any or all the claims. Accordingly, the specification and figures are to be regarded in an illustrative rather than a restrictive sense, and all such modifications are intended to be included within the scope of the claimed invention.

Unless otherwise defined, all technical and scientific terms used herein have the same meaning as commonly understood by one of ordinary skill in the art to which the claims features belong. Moreover, Applicant's inconsistent use of a term should not be construed as different terms unless defined by Applicant or the context. Likewise, it is to be understood that the phraseology and terminology employed herein are for the purpose of the description and should not be regarded as limiting. Regarding additional interpretation and construction of terms and steps herein, method steps are not in any specified order unless dictated by the context or specific wording. In addition, the use of a word in the singular form should be interpreted where the context allows or does not restrict so as to enable plurality or an “at least one” construction. Positional and directional terms described in this specification may be understood to be different than shown or described, and should not limit the variations of embodiments possible from the claimed features that a person of ordinary skill in the art would understand from the specification, figures, and claims. The term “and/or” in a list means all list items present, some list items present, or one of the list items present, unless such construction is limited by the context. “Including” shall be construed as “including but not limited to.”

### INDUSTRIAL APPLICABILITY

In addition to the goals stated above, the devices and methods herein can be used with law enforcement, mental health professionals, or other testing and interactions.

### REFERENCE NUMERALS

Police assistance device 10
Outer case 15
Display 20
Monitor Casing 25
Camera 30
Microphone 31
Light 35
Release lever 40
Handle 45
Tripod 50
Tripod Legs 60, 62, 64
Speaker 70
Speaker 80
USB (Speaker Side) 85
SD Card Slot 90
Charging Port 105
HDMI 110
USB (Back) 120
DVI Port 125
2-Channel Audio and Component Video 130
Ventilation Slots 140, 142, 144
Connections Area 160
Moving test indicator, center position 210
Moving test indicator, leftish position 220
Moving test indicator, right position 230
Processor 300
Memory 310
On-Device Storage 320
Printer 330
Video chatting Third Party 350
Base Receiving Area 400

1. A method of engaging with a suicide attempter using a police assistance device, the method comprising steps of:
providing a police assistance device to a person, the police assistance device comprising a processor, memory, a display, a camera, a microphone, and a speaker,
prompting, by the processor, the suicide attempter with predetermined messaging through the display or the speaker to follow instructions of the police assistance device; and
connecting the suicide attempter with a chosen contact or a mental health provider.

2. The method of claim 1, wherein information gathered from the suicide attempter is sent to the mental health provider.

3. A method of engaging with a person with an unknown weapon status, the method comprising steps of:
providing a police assistance device to the person, the police assistance device comprising a processor, memory, a display, a camera, a microphone, and a speaker,
prompting through the display or the speaker, by the processor, the person with predetermined messaging to follow instructions of the police assistance device;
determining, by a processor, whether the person is carrying a weapon; and
in response to a detection of a weapon, activating a weapon deterrent system.

4. The method of claim 3, wherein the activated weapon deterrent system flashes a strobe light.

5. The method of claim 3, further comprising wherein the step of determining is conducted by analyzing video footage or detecting with on-device sensors.

6. A method of claim 1, further comprising steps of:
adjusting a height of the display and the camera to be within an acceptable deviation of an eye height of the person,
prompting, by the processor, the person with predetermined messaging through the display or the speakers to follow instructions of the police assistance device;
instructing, by the processor, the person to follow using eyes of the person locations of a moving test indicator on the display;
moving, on the display, locations of the moving test indicator consistent with movements of a horizontal gaze nystagmus field sobriety test;
recording, by the camera, the locations of the moving test indicator and locations of corresponding eye movements of the person; and
storing the recorded locations of the moving test indicator and locations of the corresponding eye movements in the memory or in memory of a remotely connected system.

7. The method of claim 6 further comprising a step of analyzing, by the processor, the deviation of involuntary jerking between the locations of the moving testing indicator and the locations of the corresponding eye movements and determining if the deviation exceeds an allowable threshold.

8. The method of claim 6, wherein the deviation of involuntary jerking is measured from travel of a pupil from where the pupil should be when correspondence to occurs to where the pupil is when non-correspondence occurs between the locations of the moving test indicator and the locations of the corresponding eye movements and determining if the deviation exceeds an allowable threshold.

9. The method of claim 6, further comprising a step of admitting the recordings into evidence in a court proceeding of the person.

10. The method of claim 6, further comprising a step of admitting the recordings into evidence in a court proceeding of the person.

11. The method of claim 6, further comprising a step of transmitting the recordings onto the device or allowing access to the recording to at least one expert witness.

12. The method of claim 6, further comprising steps of:
prompting the person about which language to conduct the horizontal gaze nystagmus field sobriety test in and receiving back a selected language,
retrieving a language module corresponding to the selected language from the device or a remote system; and
changing the predetermined messaging and instruction to the selected language.

13. The method of claim 12, further comprising storing audio from the person and the selected language with the recorded locations.

14. The method of claim 6, further comprising steps of:
recording, by the microphone, audio before, during or after the camera recording of the locations of the moving test indicator and locations of the corresponding eye movements, and
storing the microphone recording in the memory or in the memory of the remotely connected system.

15. The method of claim 3, further comprising the steps of:
prompting, by the processor, a person with predetermined messaging through the display or the speakers to follow instructions of the police assistance device;
instructing, by the processor, the person to follow using eyes of the person locations of a moving test indicator on the display;
moving, on the display, the locations of the moving test indicator consistent with movements of a horizontal gaze nystagmus field sobriety test;
recording, by the camera, the locations of the moving test indicator and locations of corresponding eye movements of the person; and
storing the recorded locations of the moving test indicator and locations of the corresponding eye movements in the memory or in memory of a remotely connected system.

16. The method of claim 15, further comprising instructions of:
prompting the person about which language to conduct the horizontal gaze nystagmus field sobriety test in and receiving back a selected language,
retrieving a language module corresponding to the selected language from the device or a remote system; and
changing the predetermined messaging and instruction to the selected language.

17. The method of claim 16, further comprising instructions of storing audio from the person and the selected language with the recorded locations.

18. A police assistance device, the device comprising:
a processor;
memory storing instructions; and
the device including a display, a microphone, a camera, a speaker, and removeable storage connectors;
wherein the processor controls the device to implement the instructions in memory, the instructions comprising:
providing a police assistance device to a person, the police assistance device comprising a processor, memory, a display, a camera, a microphone, with the and a speaker,
prompting, by the processor, the person with predetermined messaging through the display or the speakers to follow instructions of the police assistance device;
connecting the person with a chosen contact or a mental health provider;
instructing a person with prerecorded messages on at least one of the display or speakers to take a HGN field sobriety test;
administering the HGN field sobriety test;
recording, in the memory, eye movements of the person during the HGN field sobriety test; and
storing the recording on the removeable storage or transmitting the recording to storage of another system.

19. The device of claim 18, the device further comprising:
a device base;
an outer case including a device base receiving area, a device base release, a display, a microphone, a camera, a speaker, and removeable storage connectors;
wherein the device base collapses and fits into the device base receiving area for stowing away the device went not in use, and
upon triggering the device base release, the device base ejects from the device base receiving area.

20. The device of claim 19, wherein the device base is a tripod with extendable legs, and the tripod fits entirely into the outer case with the extendable legs in a collapsed state.
